(19) United States
(12) Patent Application Publication
(10) Pub. No.: US 2020/0129086 A1
(43) Pub. Date: Apr. 30, 2020

(54) Sensing System for Pericardial Access

(71) Applicants: Anand GANAPATHY, Los Angeles, CA (US); David BURKLAND, Houston, TX (US); Mathews JOHN, Houston, TX (US); Brian GREET, Houston, TX (US); Mehdi RAZAVI, Houston, TX (US)

(72) Inventors: Anand GANAPATHY, Los Angeles, CA (US); David BURKLAND, Houston, TX (US); Mathews JOHN, Houston, TX (US); Brian GREET, Houston, TX (US); Mehdi RAZAVI, Houston, TX (US)

(21) Appl. No.: 16/495,933

(22) PCT Filed: Mar. 19, 2018
(86) PCT No.: PCT/US2018/023185

§ 371 Date: Sep. 20, 2019

Related U.S. Application Data
(60) Provisional application No. 62/473,907, filed on Mar. 20, 2017.

(51) Int. Cl. A61B 5/053; A61B 5/00

(52) U.S. Cl. CPC A61B 5/0538; A61B 5/6851; A61B 2505/05; A61B 2562/0209; A61B 5/6848

(57) ABSTRACT

A surgical instrument includes an impedance sensing system for monitoring the position of the tip of the surgical instrument relative to the pericardial space. The surgical instrument consists of a guidewire or needle including a conductive core terminating at a distal tip of the guidewire or needle. The distal tip has a conductive surface exposed to patient tissues and/or fluids. The impedance sensing system includes a first electrode formed by the exposed surface of the guidewire or needle, and at least a second electrode isolated from the conductive core of the guidewire or needle. The second electrode may be formed on an outer surface of the guidewire or needle, or may be a pad electrode applied to the patient skin. The impedance sensing system also includes an impedance analyzer for measuring impedance and phase using one or more frequencies.

### CROSS-REFERENCE TO RELATED APPLICATIONS

None

### BACKGROUND

This disclosure relates generally to methods and apparatus for assisting operators in various medical and interventional procedures. In particular, this disclosure relates to sensing systems for pericardial access. The sensing systems may include electrodes mounted on a surgical instrument.

Ventricular tachycardia (VT) or ventricular fibrillation causes most of the sudden cardiac deaths in the United States, at an estimated rate of approximately 300,000 deaths per year. Catheter-based ablation therapy, performed by Cardiac Electrophysiologists, has the potential to control recurrent ventricular arrhythmias and has shown to be superior to medical management. Most catheter-based ablation procedures of VT are performed via an endocardial approach to access the inner layer of the heart, with catheters introduced into the large peripheral arteries and veins and advanced to the endocardial surface of the heart. However, the most recent European Heart Rhythm Association or Heart Rhythm Society consensus document reported that the epicardial space was accessed in 17% of VT ablation procedures, based on a survey of VT tertiary referral centers. This number is expected to rise as this technique becomes more available.

The epicardium is a double-walled structure around the heart that protects and anchors the heart within the mediastinum. The pericardium is composed of two layers: the fibrous pericardium, a superficial tough, fibrous sac; and the serous pericardium, the deeper layer. The serous pericardium lines both the inner surface of the fibrous pericardium and the outer surface of the heart. These two serous layers are separated by a fluid-filled space called the pericardial space, which is the target cavity for epicardial ablation. In a normal individual, the pericardial space is filled with 15-50 ml of pericardial fluid and is less than 2.5 mm thick.

Currently, the pericardial space is accessed using operator skill. A large bore needle is inserted below the xiphoid process angled toward the head with multiple fluoroscopic images at different locations/angles used to guide the needle insertion. Once the needle is believed to be inserted into the pericardial space—often suggested by needle feedback due to the small mechanical resistance followed by a pop as the needle pierces the pericardial sac—confirmation of the location of the needle tip is typically performed using small volume contrast injections under fluoroscopy or ultrasound guidance. However, it is possible for the needle tip to migrate following injection of contrast given that both the pericardial sac and the needle tip can move several centimeters with each beat of the heart.

Once the needle appears to be in place, as confirmed for example with injection of radiopaque dye, a guidewire is advanced through the needle and into the corresponding space. After insertion of the guidewire, the needle is removed, and then a sheath can be inserted over the guidewire. When the sheath is fully inserted, the guidewire can then be removed. Then dye is once more injected into the sheath to confirm final placement is within the pericardial space.

Accessing the pericardial space is highly difficult and is a frequent source of complications, with one of the most dangerous being the needle entering the right ventricle when advanced too far. If the right ventricle is punctured, it can lead to blood accumulating in the pericardial space potentially leading to cardiac tamponade. A recent study in 2010 looking at greater than 900 consecutive patients undergoing epicardial ablation showed that 5% of cases experienced major acute complications related to epicardial access, including large pericardial effusions from right ventricular puncture requiring percutaneous or surgical drainage.

Thus, there is a need for more reliable techniques for ensuring the accessing needle has not punctured the right ventricle prior to insertion of the sheath. These techniques could significantly reduce the risk of puncture or perforation related morbidity and mortality.

### SUMMARY

The disclosure describes a sensing system, which may be used for pericardial access. The sensing system may comprise a guidewire or needle, a plurality of electrodes, and an impedance analyzer including circuitry configured to measure impedance and phase using one or more frequencies. The plurality of electrodes may be connected to the impedance analyzer by wired connectors. The wired connectors may be detachable from the impedance analyzer.

The guidewire or needle may include a conductive core terminating at a distal tip of the guidewire or needle. The guidewire or needle may further include a coating or removable lining made of electrically insulating material covering at least a portion of the length of the guidewire or needle proximate to the distal tip. The distal tip may include an exposed surface that is not covered by electrically insulating material.

The plurality of electrode may include a distal electrode formed by the exposed surface of the guidewire or needle. The plurality of electrode may also include at least one electrode isolated from the conductive core of the guidewire or needle. For example, the at least one electrode may comprise a ring or band disposed on the coating or removable lining. As used herein, a ring electrode completely encircles the coating or removable lining, and a band electrode only partially encircles the coating or removable lining. Optionally, the at least one electrode may comprise two or more electrodes located along the length of the coating or removable lining. Each of the two or more electrodes may comprise a ring or band disposed on the coating or removable lining. Alternatively or additionally, the at least one electrode may comprise a pad electrode applicable to the skin of a patient.

The impedance analyzer may comprise a signal generator operable to generate AC voltage or current signals of tunable frequencies and amplitude, circuitry to measure voltage or current, an impedance calculating circuit, and a microcontroller. The circuitry to measure voltage or current may comprise a differential amplifier. The impedance calculating circuit may be operable to calculate a position of the distal tip of the guidewire or needle. Optionally, the impedance calculating circuit may be wirelessly connected to the circuitry to measure voltage or current and/or to the signal generator.

The impedance analyzer may comprise a signal generator operable to generate AC voltage signals of tunable frequencies and amplitude, and a signal sensor operable to measure AC current signals. Conversely, the impedance analyzer may comprise a signal generator operable to generate AC current signals of tunable frequencies and amplitude, and a signal sensor operable to measure AC voltage signals. Optionally, two of the plurality of electrodes may be connected to a single terminal of the signal generator or signal sensor.

In some embodiments, the impedance analyzer may comprise a first signal generator operable to generate AC voltage or current signals of tunable frequencies and amplitude, and a second signal generator operable to generate AC voltage or current signals of tunable frequencies and amplitude. The distal electrode and a first one of the plurality of electrodes may be connected to the first signal generator. At least a second one of the plurality of electrodes, which is different from the first one of the plurality of electrodes, may be connected to the second signal generator. Optionally, other signal generators may be provided in addition to the first and second signal generators.

In some embodiments, the impedance analyzer may comprise a first signal sensor operable to measure AC voltage or current signals, and a second signal sensor operable to measure AC voltage or current signals. The distal electrode and a first one of the plurality of electrodes may be connected to the first signal sensor. At least a second one of the plurality of electrodes, which is different from the first one of the plurality of electrodes, may be connected to the second signal sensor. Optionally, other signal sensors may be provided in addition to the first and second signal sensors.

### BRIEF DESCRIPTION OF THE DRAWINGS

For a more detailed description of the embodiments of the disclosure, reference will now be made to the accompanying drawings, wherein:

FIG. 1 is a view of a surgical instrument having a plurality of electrodes, one of which being formed with the tip of the conductive core of the instrument;

FIG. 2 is a view of a detachable measuring and computing module that can be connected to the surgical instrument shown in FIG. 1;

FIG. 3 is a view of an embodiment where the surgical instrument includes a coated guidewire;

FIG. 4 is a view of an embodiment where the surgical instrument includes a coated needle;

FIG. 5 is a view of an embodiment where the surgical instrument includes a sheathed guidewire;

FIGS. 6A-6D are sagittal cross-sections of the thorax of a patient illustrating placement of a surgical instrument relative to the pericardial space using an embodiment of a sensing system in accordance with this disclosure;

FIG. 7 is a view of an embodiment where the surgical instrument includes a plurality of electrodes located along a portion of a needle covered by insulating material and/or a pad;

FIG. 7A is an enlarged view of a portion of the surgical instrument shown in FIG. 7 and illustrating an exposed surface forming a distal electrode; and

FIG. 8 is a view of an embodiment of a measuring and computing module that can be connected to the surgical instrument shown in FIG. 7.

### DETAILED DESCRIPTION

It is to be understood that the following disclosure describes several exemplary embodiments for implementing different features, structures, or functions of the invention. Exemplary embodiments of components, arrangements, and configurations are described below to simplify the disclosure; however, these exemplary embodiments are provided merely as examples and are not intended to limit the scope of the invention. Additionally, the disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various Figures. Finally, the exemplary embodiments presented below may be combined in any combination of ways, i.e., any element from one exemplary embodiment may be used in any other exemplary embodiment, without departing from the scope of the disclosure.

All numerical values in this disclosure may be exact or approximate values unless otherwise specifically stated. Accordingly, various embodiments of the disclosure may deviate from the numbers, values, and ranges disclosed herein without departing from the intended scope. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

In the following discussion and in the claims, the terms “including” and “comprising” are used in an open-ended fashion, and thus should be interpreted to mean “including, but not limited to.” Furthermore, as it is used in the claims or specification, the term “or” is intended to encompass both exclusive and inclusive cases, i.e., “A or B” is intended to be synonymous with “at least one of A and B,” unless otherwise expressly specified herein.

As one skilled in the art will appreciate, various entities may refer to the same component by different names and, as such, the naming convention for the elements described herein is not intended to limit the scope of the invention, unless otherwise specifically defined herein. Further, the naming convention used herein is not intended to distinguish between components that differ in name but not function.

The sensing systems described in this disclosure may be for use in various medical and interventional procedures. The sensing systems provide an improved means of accessing the pericardial space of patients requiring cardiac or other thoracic interventions. A purpose of the sensing systems is to assist operators in obtaining and confirming pericardial access. The sensing systems allow guiding the placement of a needle, wire, or other device to access the pericardial space. The sensing systems allow confirming the placement of said needle, wire, or other device into the pericardial space through the use of an impedance measurement. In addition, the sensing systems described herein may provide anatomic certainty that the pericardial space has been accessed while avoiding the need for fluoroscopic evaluation, thereby reducing the radioactive exposure of the patient and the operator.

An embodiment of a sensing system in accordance with this disclosure comprises the following components: 1) a surgical instrument to access the pericardial space, such as a guidewire or needle (solid or hollow) with electrodes, 2) electronics circuit that may include specialized hardware and firmware to measure impedance between these electrodes, 3) a connector between the surgical instrument and a computer, 4) computer software that is executed by the computer and is configured to assess and interpret the impedance values or other calculated values, in particular, to locate and confirm the placement of the surgical instrument within the pericardial space, and 5) a user interface that may include a display, indicators, and buttons for operators to interact with the computer software.

In some embodiments, additional electrodes may be connected to the computer to provide the reference values of impedance unique to the patient being operated. These reference values may be used to calculate normalized values of impedance as described hereinafter.

In some embodiments, an additional guidewire may be inserted through the needle or other device to extend into the pericardial space once the tip of the needle or other device has been properly positioned within the pericardial space.

The surgical instrument may comprise a guidewire (in one embodiment) or a hollow needle (in another embodiment) or a solid needle (in yet another embodiment) that is electrically conductive. The surgical instrument has a distal tip (that, in use, is the farthest from the operator) which is exposed to tissues and fluids of the patient body. The exposed tip acts as a distal electrode. The rest of the surgical instrument is electrically isolated from the tissues and fluids of the patient body with a coating or a removable lining. Either one or a plurality of electrodes may be embedded in the coating or removable lining to provide at least one bipolar set of electrodes suitable for impedance measurement therebetween. These electrodes may be coupled to insulated wires that run along the insulated portion of the surgical instrument toward the proximal end of the surgical instrument. The wires and the surgical instrument are connected to an impedance analyzer.

In some embodiments, the surgical instrument includes a guidewire and a plurality of electrodes that may be connected to an impedance analyzer. The impedance analyzer is used to measure impedance values. These impedance values may then be provided to the operator either directly or after processing with an algorithm of the computer software, allowing for a determination of the location of the guidewire without requiring fluoroscopic imaging or ultrasonic real-time guidance.

In some embodiments, the surgical instrument includes a needle with a plurality of electrodes that may be connected to an impedance analyzer. The impedance analyzer is used to measure impedance values. These impedance values may then be provided to the operator either directly or after processing with an algorithm of the computer software, allowing for a determination of the location of the needle without requiring fluoroscopic imaging or ultrasonic real-time guidance.

In some embodiments, the surgical instrument includes an electrically conductive guidewire that is coated with electrically insulating material. The coated guidewire may attain a diameter that is similar to that of other commercially available epicardial access guidewires. The guidewire is preferably flexible enough to navigate safely within the pericardial space, and firm enough to provide support for guiding an intrapericardial sheath. The guidewire coating leaves the distal tip (that is farthest from the operator) uncovered and exposed to tissues or fluids in the patient body. Along the length of the coated wire, and close to the distal tip of the wire, one or more electrically conductive bands are provided. These bands form electrodes that are preferably electrically insulated from each other and from the guidewire. The bands are preferably coupled to dedicated wires that are disposed along the length of the guidewire. The wires are optionally embedded within the insulating material of the coating. These dedicated wires and the conductive core of guidewire are connected to an impedance analyzer. These dedicated wires and the guidewire are optionally detachable from the impedance analyzer. The connection may include standard pins and sockets or other types of connections.

Impedance measurements may be performed between the distal tip of the surgical instrument and a single electrode or multiple electrodes. In one embodiment, a tunable current may be circulated into the tissue of the patient and the resultant potential difference between the distal tip of the surgical measurement and another electrode may be measured. The measured potential difference may then be used to calculate impedance values as a function of time. In another embodiment, a tunable voltage signal may be applied across the tissue of the patient and the resultant current may be measured to calculate the impedance. In another embodiment, the impedance may be measured by changes in the voltage signal.

In some embodiments, the impedance analyzer may include a signal generator capable of generating AC voltage signals of tunable frequencies and amplitude, a tunable current generator circuit such as a Howland's circuit, a differential amplifier, an analog-to-digital converter and a microcontroller, among other components which may be used to measure impedance values.

In some embodiments, the impedance analyzer comprises a signal generator, front-end signal conditioning circuit, and voltage/current measuring circuit. The computer software, implemented on a logic board, is configured to calculate impedance from data provided by the voltage/current measuring circuit.

A detachable connection is preferably provided between the wire ends coupled to the electrodes or coupled to the instrument conductive core and the impedance analyzer. One example connection includes a standard connection such as pin socket connectors allowing a stable connection. Another example connection includes conductive rings or bands similar to electrodes provided on the proximal end of the surgical instrument. A female connector coupled to the electronic circuit also includes corresponding conductive rings or bands. In this embodiment, the female connector slides over the proximal end of the surgical instrument, and contacts between the conductive rings or band are established.

The computer software includes algorithms that may facilitate navigation and/or position confirmation of the tip of the surgical instrument relative to the pericardial space. Accordingly, the impedance values are monitored during insertion of the medical instrument into the body of the patient. The impedance values may be either directly displayed to an operator, in the form of absolute impedance values, or in the form of relative impedance values, which have been normalized by a reference value unique to the patient being operated. The impedance values, either absolute or relative, may also be displayed graphically, such as by colors, shapes of varying sizes, or other graphical representations. The impedance values may also be used to provide a map of the access space enabling visualization of the space.

In one example, the absolute impedance values may be used directly by the operator to determine if the surgical instrument is in the pericardial space, or has perforated the right ventricular wall. Accordingly, the absolute value of the impedance (variable X) may be used to determine the position of the tip of the surgical instrument relative to the pericardial space.

In another example, reference impedance values (“control” impedances) may be collected at different locations on the patient using electrodes of the surgical instrument or other electrodes placed within the patient's IVC. The absolute impedance values may be normalized by the reference impedance values (variable Y), expressed as an impedance ratio (X/Y). Impedance ratios within defined ranges may indicate the position of the tip of the surgical instrument, whether within the pericardial space, the intramural right ventricular space, or the anterior mediastinal space.

In other examples, the impedance values may be used to indicate the position of the tip of the surgical instrument relative to an organ other than the heart, such as the liver.

In yet another example, impedance values and phase values at the different frequencies are used to calculate the position of the tip of the surgical instrument relative to the pericardial space.

In addition, the position of the tip of the surgical instrument relative to the pericardial space may be confirmed by monitoring electrocardiogram signal detected by one or more electrodes.

A user interface may be used to notify the operator of the absolute impedance values, the normalized impedance values, or any combination thereof. For example, a visual display, auditory sounds, etc. may be used. The user interface may also permit selection of current density, voltage, frequency, a pair of electrodes to be monitored, etc.

Pre-clinical testing performed in the ovine and porcine model have demonstrated the feasibility of proper positioning of a catheter by measurement of the impedance between the bipolar set of electrodes provided at the distal tip of the finder needle. The data collected during pre-clinical testing show a substantial, consistent difference in impedance between the anterior mediastinal compartment, the pericardial space, and the right ventricular space.

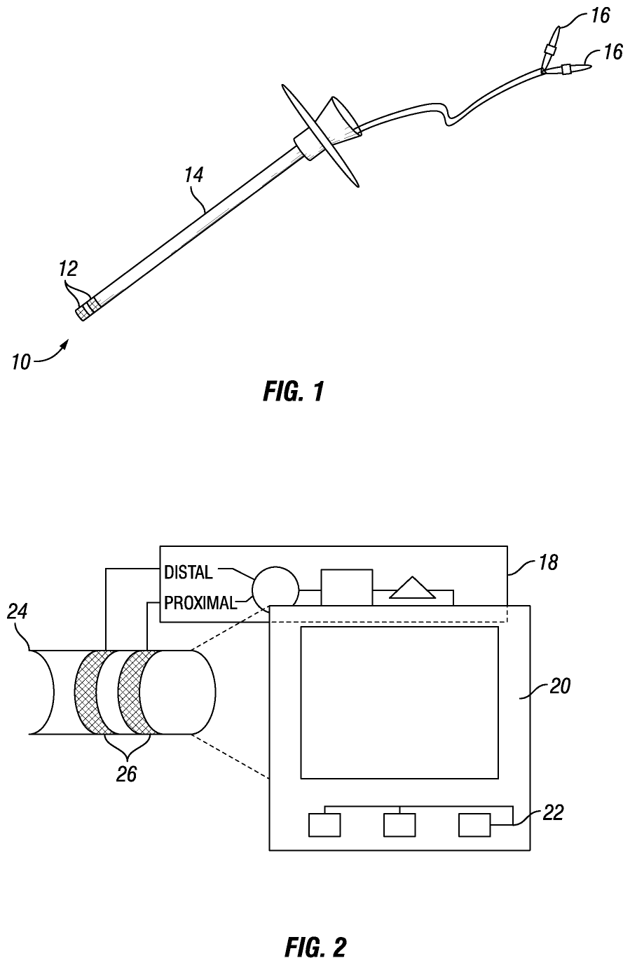

Referring to FIG. 1, the distal end 10 (i.e., the end inserted into the patient) of an epicardial access instrument 14 is fitted with a bipolar set of electrodes 12 to measure an impedance between a distal electrode and another electrode. The distal electrode is formed with the tip of the conductive core of the instrument 14 and an insulating coating. The insulating coating supports the other electrode and a conductive path coupled to the other electrode. In addition, the proximal end of the instrument 14 is provided with a connection 16.

In some embodiments, a plurality of independent electrodes are supported on the insulating coating or removable lining.

In some embodiments, the instrument may be a guidewire or a needle (hollow or solid) or another device.

The connection 16 between the bipolar set of electrodes and the measuring and computing module may be accomplished via contacts, or via contactless couplings.

Turning to FIG. 2, a detachable measuring and computing module 22 contains an impedance analyzer 18 and a user interface 20. The measuring and computing module 22 includes a connection 24 having a pair of conductive contacts 26 that may then be connected to the connection 16 of the instrument shown in FIG. 1.

A sensing system comprising the instrument 14 fitted with the bipolar set of electrodes 12 and the measuring and computing module 22 may be used to determine a location of the distal end 10 of the instrument 14 relative to the pericardial space of a patient. The sensing system may also be used to confirm placement of the distal end 10 of the instrument 14 within the pericardial space. The instrument 14 may be used like a standard wire in cardiac or other thoracic interventions when the measuring and computing module 22 is detached.

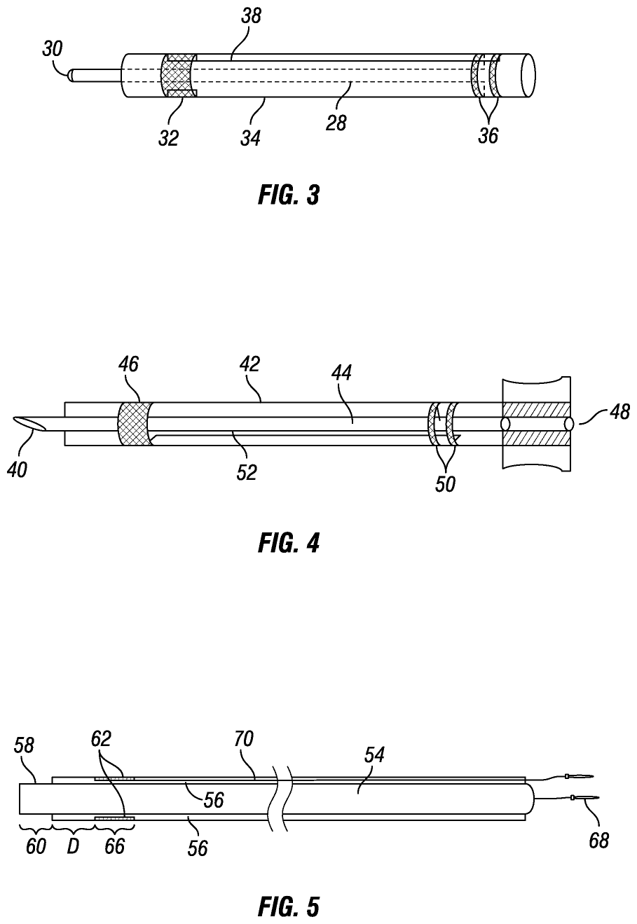

Turning to FIG. 3, an electrically conductive core 28 of a guidewire is coated with an electrically insulating material 34. A distal tip 30 remains uncoated or exposed to function as the distal electrode. Another electrode may consist of a singular ring or band 32 of electrically conductive material. The electrically conductive core 28 is electrically insulated from the other electrode by the coating of insulating material 34.

The electrically conductive core 28 serves as an electrical path to the distal electrode provided by the uncoated distal tip 30. The electrically conductive core 28 can be coupled to one of a pair of conductive contacts 36 near the proximal end of the guidewire. An insulated wire 38 is coupled to the ring or band 32, and runs down the length of the insulating material 34 to the proximal end of the guidewire. The insulated wire 38 can be coupled to another one of the pair of conductive contacts 36.

The coated guidewire may be attached to the measuring and computing module 22 shown in FIG. 2. The pair of conductive contacts 36 is may be connected to the corresponding pair of conductive contacts 26. Impedance between the distal electrode provided by the uncoated distal tip 30 and the other electrode provided by the ring or band 32 may be continuously monitored with the measuring and computing module 22.

In some embodiments, multiple rings or bands of electrically conductive material, similar to the ring or band 32, may be disposed along the length of the coating away from the uncoated distal tip 30.

Turning to FIG. 4, a hollow needle has a bore 48 extending along its length. A distal tip 40 remains uncoated or exposed and forms a distal electrode. Electrically insulating material 42 forms a coating surrounding the conductive material of the hollow core 44. The ring or band 46 forms the other electrode of a set of bipolar electrodes. The other electrode is coupled via a wire 52 to one of a pair of the conductive contacts 50. The conductive contacts 50 may be connected to the measuring and computing module 22 shown in FIG. 2.

In some embodiments, the needle may be solid and may not have a bore. In these embodiments, the needle still has a perforating sharp tip.

In some embodiments, multiple rings or bands of electrically conductive material, similar to the ring or band 46, may be disposed along the length of the coating away from the uncoated distal tip 40.

Turning to FIG. 5, an industry standard guidewires 54 is inserted into a removable lining 56 made of electrically insulating polymer. For example, guidewires 54 may have a diameter between 0.014 inch and 0.035 inch, which is representative of commercially standard diameters of guidewires for epicardial access. A distal tip 58 extends from the removable lining 56 and remains exposed to function as a distal electrode. For example, a length 60 of exposed guidewire may be approximated 0.2 inch. The other electrode may include a singular ring or band 62 of electrically conductive material that can be embedded into the removable lining 56. For example, a width 66 of the ring or band 62 may be approximately 0.2 inch. The ring or band 62 may be located a distance D away from the distal tip 58. The distance D may be approximately 0.2 inch.

The distal electrode formed by the uncoated distal tip 58 is electrically coupled to one of a pair of connectors 68 located at the proximal end of the guidewire 54. The other electrode formed by the ring or band 62 can be coupled to the other of the pair of connectors 68 via a wire 70 which may also be embedded in removable lining 56. The wire 70 runs along the length of the removable lining 56 to the proximal end of the guidewire 54.

In the example shown, the pair of connectors 68 includes pins that may be connected to corresponding sockets provided with the measuring and computing module 22 shown in FIG. 2.

In other embodiments, the removable lining 56 may include a plurality of rings or bands similar to ring or band 62 that are embedded into and distributed along the length of the removable lining 56.

In some embodiments, the removable lining 56 may be removed from around the guidewire 54 after the guidewire 54 has been inserted into the patient.

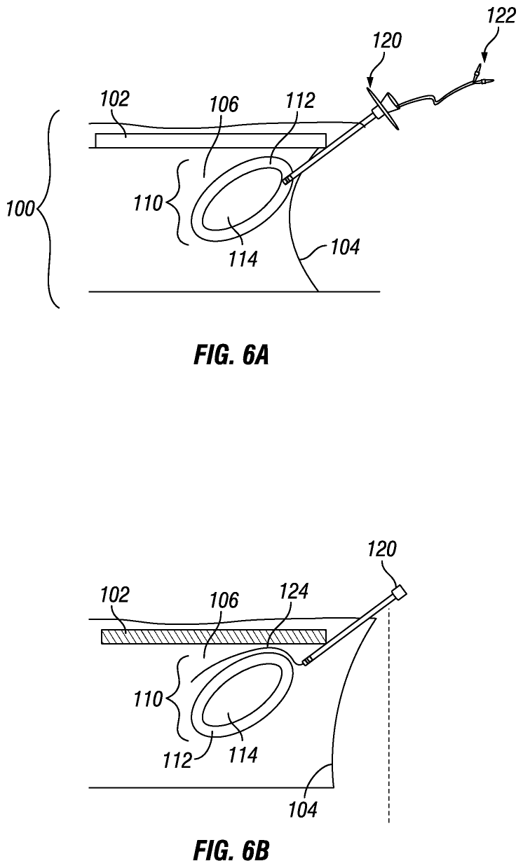

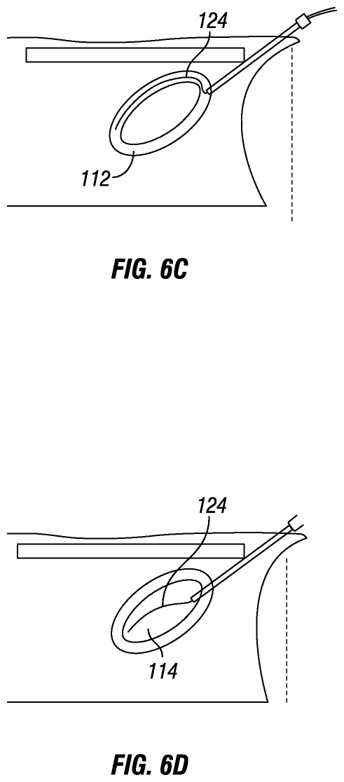

FIGS. 6A-6D are sagittal cross-sections of the thorax 100 of a patient. The sternum 102, diaphragm 104, anterior mediastinal compartment 106, heart 110, pericardial space 112, and right ventricular intramural space 114 are shown.

FIGS. 6A-6D illustrate the relative positioning of a finder needle 120 in accordance with one example embodiment. In this example embodiment, the finder needle 120 is provided with a bipolar set of electrodes at its distal tip, as described herein. A pair of connection pins 122 are provided at the proximal end of the finder needle 120. Each connection pin of the pair of pins 122 is electrically coupled to corresponding electrodes in the bipolar set provided at the distal tip of the finder needle 120. In use, the pair of connection pins is connected to the measuring and computing module 22 shown in FIG. 2 to measure an impedance across the bipolar set of electrodes.

In FIG. 6A, the needle 120 is illustrated positioned in its proper position within the pericardial space 112 according to the standard percutaneous subxiphoid approach.

In FIG. 6B, the finder needle 120 is illustrated improperly positioned within the anterior mediastinal compartment 106. In those situations, a pericardial guidewire introduced through the finder needle 120 may accidentally be inserted into the anterior mediastinal compartment 106. These situations may be avoided by continuously monitoring the impedance across the bipolar set of electrodes of the finder needle 120. Improper positioning of the finder needle 120 may be detected by a larger than expected measurement of the impedance between the bipolar set of electrodes provided at the distal tip of the finder needle 120.

In FIG. 6C, the finder needle 120 and the pericardial guidewire 124 are illustrated properly inserted within the pericardial space 112. The proper positioning of the finder needle 120 may be confirmed by measurement of the impedance between the bipolar set of electrodes provided at the distal tip of the finder needle 120 within an expected range.

FIG. 6D shows the finder needle 120 improperly positioned within the right ventricle, and the pericardial guidewire 124 accidentally inserted into the right ventricular intramural space 114. Such improper positioning of the finder needle 120 may be avoided by detection of a lower than expected measurement of the impedance between the bipolar set of electrodes provided at the distal tip of the finder needle 120.

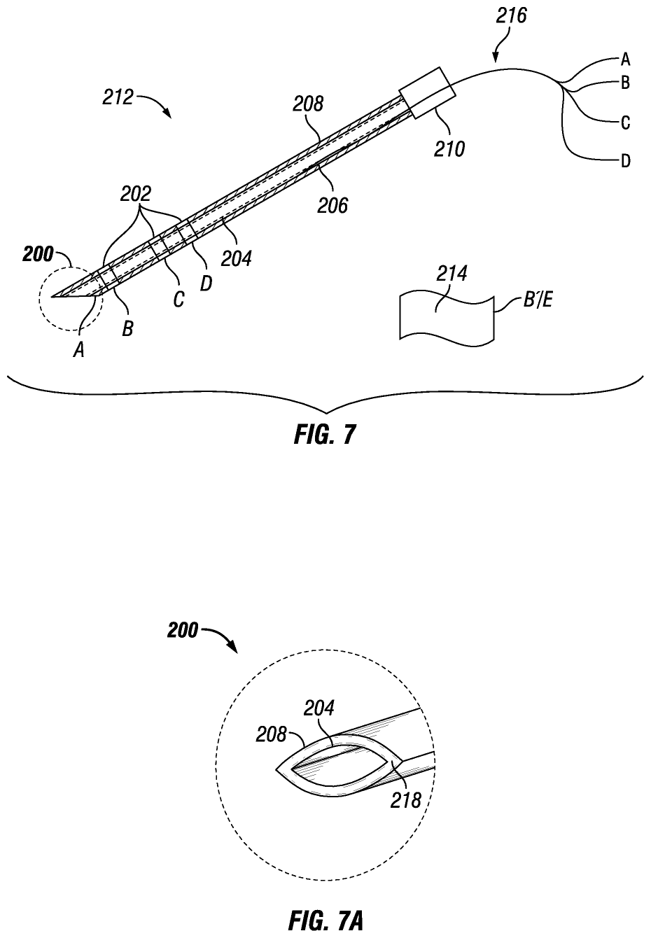

FIG. 7 shows a needle 212 that includes a metallic hollow core 206. The core 206 terminates at a distal tip 200, and is coupled at a proximal end to a plastic or metallic hub 210. The needle 212 also includes an inner surface insulation 204, and an outer surface insulation 208. Either or both of the inner surface insulation and the outer surface insulation may be implemented with a coating or removable lining made of electrically insulating material.

The distal tip 200 includes an exposed surface 218 of the metallic hollow core 206, which form a distal electrode, which is labeled A. A bottom view of the exposed surface 218 is illustrated in FIG. 7A. Two or more ring electrodes 202 are located along the length of the outer surface insulation 208. In the example shown, the ring electrodes 202 are labeled B, C, and D. Each of the ring electrodes 202 is connected to an impedance analyzer with wires 216.

FIG. 7 also shows a pad electrode 214 applicable to the skin of a patient. The pad electrode 214 may be provided instead of the ring electrodes 202, in which case it may be labeled B′, or in addition to the ring electrodes 202, in which case it may be labeled E. The pad electrode 214 is also connected to an impedance analyzer.

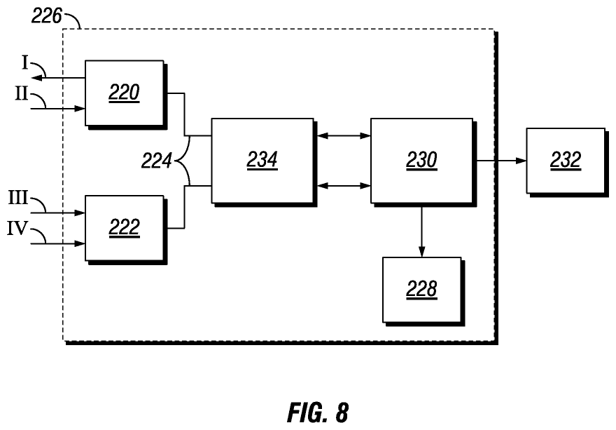

FIG. 8 shows an embodiment of a measuring and computing module comprising an impedance analyzer 226 connected to an external monitor 232. The impedance analyzer includes at least one signal generating circuit 220, which has a pair of terminals, labeled I and II, and at least one signal measuring circuit 222, which has a pair of terminals, labeled III and IV. More than one signal generating circuit may be provided. For example, another signal generating circuit (not shown) may have a pair of terminals labeled I′ and II′, and another signal measuring circuit (not shown) may have a pair of terminals labeled III′ and IV′. The terminals can be selectively connected, for example, to the electrodes A, B, C, D, and B′ or E that have been illustrated in FIG. 7. Various connections may be available, as further discussed herein with respect to the description of Table 1.

The signal generating circuit 220 and the signal measuring circuit 222, and any additional generating and or measuring circuit(s), are connected via connections 224, which may be wired or wireless connections, to an impedance calculating circuit of the impedance analyzer 226. The impedance calculating circuit—which includes all the other electronics, such as a controller 230 configured to drive the signal generating 220 and the signal measuring circuit 222, a data logging circuit 228, and additional circuit 234, computes the impedance magnitude(s) and/or phase(s) and either displays them or communicates them externally to the monitor 232 or a printer.

In some embodiments, the signal generating circuit 220 is implemented with a current source, and is combined with a voltage measurement provided by the signal measuring circuit 222. Conversely, the signal generating circuit 220 is implemented with a voltage source, and is combined with a current measurement provided by the signal measuring circuit 222. Thus, different pairs of electrodes can be used for the purpose of either transmitting the generated voltage and/or current and measuring the subsequent current and/or voltage, respectively.

Moreover, impedance can be measured from any number of electrodes, with the electrodes in a bipolar (2-), tripolar (3-), tetrapolar (4-) electrode configuration, a combination of these configurations, or other configurations if more electrodes are provided. Any set of electrodes that measure impedance can have one or more electrodes sourcing alternating current/voltage and one or more electrodes measuring the corresponding voltage/current. For example, in a 3-electrode configuration, only one electrode may be measuring voltage, and the other two electrodes may be sourcing current or vice-versa. Table 1 illustrates a non-exclusive list of configurations.

TABLE 1

| | SOURCES | | | | MEASUREMENTS | | | |
|---|---|---|---|---|---|---|---|---|
| | I | II | I′ | II′ | III | IV | III′ | IV′ |
| 4-poles | A | D | | | B | C | | |
| 2-poles simultaneous | A&C | B&D | | | A&C | B&D | | |
| 2-poles alternate | A | B | C | D | A | B | C | D |

As exemplified in Table 1, four electrodes may be configured as two 2-electrode configurations. Each 2-electrode configuration can source out an alternating current and/or voltage and also measure the corresponding voltage and/or current. Further, impedance may be monitored from the two 2-electrode configurations simultaneously or alternatingly. In the alternating case, each 2-electrode configuration is not active for a set period of time.

In case of using multiple electrodes, such as two 2-electrode configurations, the generated signals (current or voltage source) for both 2-electrode configurations maybe be multiplexed, in which case the signal generating circuit 220 may be provided with additional circuitry. Alternatively, the generated signals (current or voltage source) can be connected in series (for current source) or in parallel (for voltage source). The signal measuring circuit 222 may be configured in the same manner. While Table 1 shows at most two signal generators and at most two signal sensors, other embodiments may include more than two signals generators and/or more than two signal sensors.

While the disclosure is susceptible to various modifications and alternative forms, specific embodiments thereof are shown by way of example in the drawings and description. It should be understood, however, that the drawings and detailed description thereto are not intended to limit the claims to the particular form disclosed, but on the contrary, the intention is to cover all modifications, equivalents, and alternatives falling within the scope of the claims.

1. A sensing system for indicating a position of a tip of a surgical instrument, comprising:
a needle including a conductive core terminating at a distal tip of the needle, wherein the distal tip includes an exposed surface that is not covered by electrically insulating material, wherein the needle further includes a coating or removable lining made of electrically insulating material covering at least a portion of the length of the needle proximate to the distal tip;
a plurality of electrodes including:
a distal electrode formed by the exposed surface of the needle; and
at least one electrode isolated from the conductive core of the needle, wherein the at least one electrode comprises a ring or band disposed on the coating or removable lining; and
an impedance analyzer including circuitry configured to continuously measure impedance and phase using one or more frequencies,
wherein the plurality of electrodes are connected to the impedance analyzer by wired connectors.

2. (canceled)

3. (canceled)

4. The sensing system of claim 1, wherein the plurality of electrodes further includes a pad electrode applicable to the skin of a patient and connected to the impedance analyzer.

5. The sensing system of claim 1, wherein the at least one electrode comprises two or more electrodes located along the length of the coating or removable lining and connected to the impedance analyzer.

6. The sensing system of claim 5, wherein each of the two or more electrodes comprises a ring or band disposed on the coating or removable lining.

7. The sensing system of claim 5, wherein the impedance analyzer comprises:
a first signal generator operable to generate AC voltage or current signals of tunable frequencies and amplitude, wherein the distal electrode and a first one of the plurality of electrodes are connected to the first signal generator; and
a second signal generator operable to generate AC voltage or current signals of tunable frequencies and amplitude, wherein at least a second one of the plurality of electrodes, which is different from the first one of the plurality of electrodes, is connected to the second signal generator.

8. The sensing system of claim 5, wherein the impedance analyzer comprises:
a first signal sensor operable to measure AC voltage or current signals, wherein the distal electrode and a first one of the plurality of electrodes are connected to the first signal sensor; and
a second signal sensor operable to measure AC voltage or current signals, wherein at least a second one of the plurality of electrodes, which is different from the first one of the plurality of electrodes, is connected to the second signal sensor.

9. The sensing system of claim 5, wherein the impedance analyzer comprises:
a signal generator operable to generate AC voltage signals of tunable frequencies and amplitude;
a signal sensor operable to measure AC current signals, and
wherein two of the plurality of electrodes are connected in parallel to the signal generator.

10. The sensing system of claim 5, wherein the impedance analyzer comprises:
a signal generator operable to generate AC current signals of tunable frequencies and amplitude;
a signal sensor operable to measure AC voltage signals, and
wherein two of the plurality of electrodes are connected in parallel to the signal sensor.

11. The sensing system of claim 1, wherein the wired connectors are detachable from the impedance analyzer.

12. The sensing system of claim 1, further comprising a computer executing computer software and a user interface, the computer software being capable of determining a position of the distal tip of the needle.

13. The sensing system of claim 1, wherein impedance analyzer includes:
a signal generator operable to generate AC voltage or current signals of tunable frequencies and amplitude;
circuitry to measure voltage or current;
an impedance calculating circuit; and
a microcontroller.

14. The sensing system of claim 13, wherein the circuitry to measure voltage or current includes a differential amplifier.

15. The sensing system of claim 13, wherein the impedance calculating circuit is wirelessly connected to the circuitry to measure voltage or current, or wirelessly connected to the signal generator.

16. The sensing system of claim 12, wherein the computer software includes algorithms to collect at least one reference impedance value using the plurality of electrodes, to normalize the impedance and phase continuously measured by the impedance analyzer by the at least one reference impedance value as an impedance ratio, and to indicate whether the impedance ratio is within predefined ranges.
